(19) United States
(12) Patent Application Publication
(10) Pub. No.: US 2022/0309043 A1
(43) Pub. Date: Sep. 29, 2022

(54) DATA QUALITY CHECKING BASED ON DERIVED RELATIONS BETWEEN TABLE COLUMNS

(71) Applicant: KONINKLIJKE PHILIPS N.V., EINDHOVEN (NL)

(72) Inventors: Johannes Henricus Maria KORST, EINDHOVEN (NL); Serverius Petrus Paulus PRONK, VUGHT (NL); Mauro BARBIERI, EINDHOVEN (NL); Marc André PETERS, VELDHOVEN (NL); Qi GAO, EINDHOVEN (NL); Antonio PERRONE, EINDHOVEN (NL)

(21) Appl. No.: 17/615,641

(22) PCT Filed: Jun. 19, 2020
(86) PCT No.: PCT/EP2020/067198

§ 371 Date: Dec. 1, 2021

Related U.S. Application Data
(60) Provisional application No. 62/866,679, filed on Jun. 26, 2019.

(51) Int. Cl. G06F 16/21; G06F 16/215; G06F 16/23

(52) U.S. Cl. CPC G06F 16/211; G06F 16/215; G06F 16/2365; G16H 10/60

(57) ABSTRACT

In an electronic data processing method, a first column and a second column of at least one table are identified which have a one-to-one or one-to-many relationship with fewer than a threshold number of violations of the one-to-one or one-to-many relationship. On a display, possible data errors in the at least one table corresponding to the violations of the one-to-one or one-to-many relationship are indicated. The identifying of the violations may include generating a weighted bipartite graph representing the first and second columns, and detecting the violations using the bipartite graph. The method may further include displaying, on the display, a user interface whereby a user accepts or rejects each indicated violation of the one-to-one or one-to-many relationship.

### FIELD

The following relates generally to the information acquisition, transmission, and processing arts, information quality control arts, information logging arts, medical imaging device machine and service logging arts, patient monitoring arts, and related arts.

### BACKGROUND

Information for a given task may be acquired via numerous pathways, such as manual data entry, reading sensor devices, generating data by electronic computations, reading data from a data storage device, various combinations thereof, or so forth. Such data can be inaccurate for numerous reasons, such as data entry errors, sensor glitches or failures, values dropped or corrupted during electronic data transmission, reading data from an incorrect data file, or so forth. It is known to perform automated data checking to flag some such errors, for example an exact zero value may be flagged as suspicious if the source is such that it should not generate exact zero values, or data of a wrong data type may be flagged (e.g. an integer when the value should be a floating point value), or other similar data checks. However, these automated checks can potentially miss many errors that do not fail any of these limited quality control checks. A human domain expert could detect many other errors, but manual review of large quantities of data may be impractical due to the sheer data volume and/or limited availability of human domain experts with requisite special expertise.

Accuracy of information is important in many applications. As one illustrative example, modern medical imaging devices are complex systems with tens of thousands of interacting components, and are commonly designed to incorporate control and monitoring electronics which generates large volumes of machine log data. Such systems are serviced on routine and as-needed bases, and service log data are generated during servicing by manual data entry performed by the service engineer and possibly by other mechanisms such as records of an online parts ordering system, service log data automatically generated by the imaging device electronics, and/or so forth. Inaccuracies in machine or service log data can lead to increased cost due to unnecessary or ineffective servicing, and in some cases can lead to degradation of the image quality of acquired clinical images which can have adverse impact on the clinical services provided by the medical imaging device. Yet the volume and domain specificity of much of the content of the machine and service logs makes detection of errors difficult.

As another illustrative example, modern electronic medical record (EMR), electronic health record (EHR), cardiovascular information systems (CVIS), and other such patient data recordation systems present high complexity. A single multifunction patient monitor may acquire numerous vital signs (for example, heart rate, respiration rate, blood oxygenation level, capnography data, one or more types of blood pressure, electrocardiogram, electroencephalogram, or so forth) and possibly other patient monitoring data such as intravenous therapy flow rate. These data may be acquired intermittently or continuously (or, more precisely, at a high sampling rate). These data are subject to errors due to sensor glitches from patient movements or other problems with the patient coupling, and due to corruption during transmission from the patient monitor to the server computer maintaining the EHR. Other patient data in an EMR may include demographic information manually entered by clerical staff and/or retrieved from other database systems such as those of another hospital or an insurance company, and these data are prone to data entry errors, data retrieval errors, or the like. Errors in patient data can have adverse consequences for the patient's clinical treatment and for ancillary activities such as insurance billing. Yet the volume and domain specificity of much of the content of an EMR makes detection of errors difficult.

These are merely some illustrative examples, and it will be appreciated that accuracy of information is similarly important in many other fields in the medical domain as well as in numerous fields beyond the medical domain.

The following discloses certain improvements.

### SUMMARY

In some non-limiting illustrative embodiments disclosed herein, an electronic data processing system comprises an electronic processor and a non-transitory storage medium storing instructions readable and executable by the electronic processor to perform an electronic data processing method. The method includes: performing pairwise comparisons of columns of at least one table to associate a first column and a second column with a one-to-N relationship by detecting violations of the one-to-N relationship for data of the first and second columns and determining that a count of the violations of the one-to-N relationship for the first and second columns is less than a threshold; and indicating, on a display, possible data errors corresponding to the violations of the one-to-N relationship for data of the first and second columns. The performing and the indicating are performed for the one-to-N relationship being a one-to-one relationship and/or for the one-to-N relationship being a one-to-many relationship.

In some non-limiting illustrative embodiments disclosed herein, a non-transitory storage medium stores instructions that are readable and executable by an electronic processor to perform an electronic data processing method comprising: identifying a first column and a second column of at least one table which have a one-to-one or one-to-many relationship with fewer than a threshold number of violations of the one-to-one or one-to-many relationship; and indicating, on a display, possible data errors in the at least one table corresponding to the violations of the one-to-one or one-to-many relationship.

In some non-limiting illustrative embodiments disclosed herein, an electronic data processing method is disclosed. Using an electronic processor, a first column j and a second column j′ of at least one table are identified which have a mostly one-to-N relationship, where the entry in row i and column j of the table is denoted by t(i,j). On a display, possible data errors in the at least one table corresponding to violations of the identified one-to-N relationship are indicated. The one-to-N relationship is a one-to-one relationship defined as t(i,j)=t(i′,j) if and only if t(i,j′)=t(i′,j′) or a one-to-many relationship from column j to column j′ defined as t(i,j)=t(i′,j) if t(i,j′)=t(i′,f) but not necessariy vice versa, where i and i′ are different rows of the at least one table. The same can be defined for columns from different tables, say column j from table t and column j′ from table t′, provided that a one-to-one relationship is known between the rows of the two tables.

One advantage resides in providing an electronic data processing system with information-agnostic data quality checking.

Another advantage resides in providing an electronic data processing system with data quality checking that makes minimal a priori assumptions.

Another advantage resides in providing an electronic data processing system with data quality checking that does not rely upon a priori information beyond the data itself.

Another advantage resides in providing an electronic data processing system with data quality checking operating on one-to-one relations between different columns of data in the same table or in different tables.

Another advantage resides in providing an electronic data processing system with data quality checking operating on one-to-many relations between different columns of data in the same table or in different tables.

Another advantage resides in providing an electronic medical data processing system with one or more of the foregoing benefits.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates an electronic data processing system including data quality checking based on derived one-to-one and/or one-to-many relations.

FIG. 2 diagrammatically illustrates a table of data which is used as an illustrative example of data quality checking based on derived one-to-one and/or one-to-many relations suitably performed by the system of FIG. 1.

FIG. 3 diagrammatically illustrates a weighted bipartite graph representing columns identified as j=1 and j=2 in the table of FIG. 2, which illustrates data quality checking based on derived one-to-one and/or one-to-many relations suitably performed by the system of FIG. 1.

FIG. 4 diagrammatically illustrates a weighted bipartite graph representing columns identified as j=1 and j=3 in the table of FIG. 2, which illustrates data quality checking based on derived one-to-one and/or one-to-many relations suitably performed by the system of FIG. 1.

FIG. 5 diagrammatically illustrates identification of corresponding rows of the first and second tables, which may be used to perform data quality checking based on derived one-to-one and/or one-to-many relations between columns of different tables.

### DETAILED DESCRIPTION

Data quality assessment techniques disclosed herein provide for detecting data errors in an information-agnostic fashion; that is, without taking into consideration the information represented by the data values. The disclosed approaches assumes data in a table format in which the rows correspond to different entities (e.g., different patients, different medical imaging devices, or so forth) while the columns correspond to data fields. Hence, entry t(i,j) represents the value of the data field represented by j for entity i. The data may be stored in a single table, or may be stored in two or more different tables so long as corresponding rows can be identified, for example based on an index value identifying the entity (such as a machine identifier in the case of medical imaging device machine logs, a patient name or patient identifier, PID, in the case of patient data, or so forth). This is the extent of a priori assumptions—there are no further assumptions made regarding the informational content of the various columns j.

In description herein, tabular data is described in terms of “rows” corresponding to different entities, and “columns” corresponding to different data fields. It will be appreciated that other analogous terms may be employed in a specific implementation or description, for example a “row” may alternatively be referred to as a “record”, a “tuple”, a “vector”, or other analogous phraseology—all such analogous phraseology is to be understood as being encompassed by the term “row” as used herein. Similarly, a “column” may be alternatively referred to as a “field”, a “parameter”, a “property”, an “attribute”, a “vector element”, or other analogous phraseology— all such analogous phraseology is to be understood as being encompassed by the term “column” as used herein. Still further, the term “table” may alternatively be referred to as a list of tuples, a set of vectors, or so forth—again, all such analogous phraseology is to be understood as being encompassed by the term “table” as used herein.

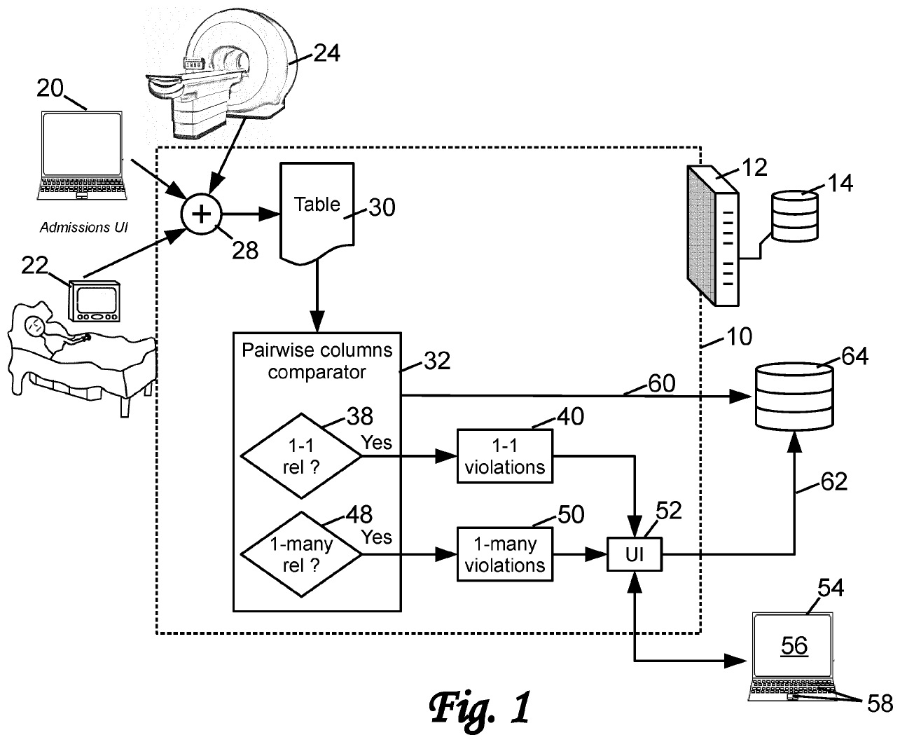

With reference to FIG. 1, an illustrative electronic data processing system 10 is shown, which includes data quality checking based on derived (mostly) one-to-one and/or (mostly) one-to-many relations as disclosed herein. The electronic data processing system 10 is implemented by at least one electronic processor 12, such as an illustrative server computer, and a non-transitory storage medium 14 that stores instructions readable and executable by the electronic processor 12 to implement the electronic data processing system 10. While a server computer 12 is illustrated, more generally the electronic processor 12 may be a single server computer (as shown), a plurality of interconnected server computers cooperatively reading and executing the instructions stored on the non-transitory storage medium 14 (for example, interconnected as a server cluster, an ad hoc cloud computing resource, or so forth), a desktop computer, a notebook computer, a control computer or programmable electronic controller of a medical imaging device, various combinations thereof, and/or so forth. The non-transitory storage medium 14 may be variously implemented, for example as a hard disk or other magnetic storage medium, a solid state drive or other electronic storage medium, an optical disk or other optical storage medium, a combination of hard disks and/or solid state drives and/or optical disks such as a Redundant Array of Independent Disks (RAID), and/or so forth.

The electronic data processing system 10 receives data for processing from one or more sources, such as an illustrative data entry terminal 20 (for example, used to enter patient information such as name, age, gender, ethnicity, reason for admission, and so forth), a patient monitor 22 (e.g. an illustrative bedside multifunction monitor such as may be provided in a hospital room, or a more specific patient monitor generating specific data such as an electrocardiogram, an intravenous infusion pump generating flow rate data, various combinations thereof, and/or so forth), a medical imaging device 24 generating automatically machine logs and/or automatically and/or manually generated service logs, various combinations thereof, and/or so forth. The illustrative input sources 20, 22, 24 are merely examples, and a given embodiment of the electronic data processing system 10 may include some subset of these inputs, additional inputs, multiple instances of a given data type of input, and/or so forth. For example, if the electronic data processing system 10 is an Electronic Medical Record (EMR) (this term being broadly used herein to encompass any electronic data processing system for recording patient data, such as an electronic health record (EHR), cardiovascular information systems (CVIS), or so forth) then the inputs may include the data entry terminal 20 used for patient admissions and a large number of patient monitors 22. As another example, if the electronic data processing system 10 is a call center for a medical imaging devices manufacturer then the inputs may include a fleet of medical imaging devices 24 such as magnetic resonance imaging (MRI) scanners, computed tomography (CT) scanners, positron emission tomography (PET) scanners, gamma cameras used for single photon emission computed tomography (SPECT) imaging, hybrid medical imaging devices such as PET/CT scanners, various combinations thereof, and/or so forth. It will be appreciated that the received data may be of a wide range of different data types (e.g. free-form text, integers, floating point values, logical data fields, structured data fields having a closed set of possible values for example, generated by a drop-down list of a graphical user interface, and/or so forth).

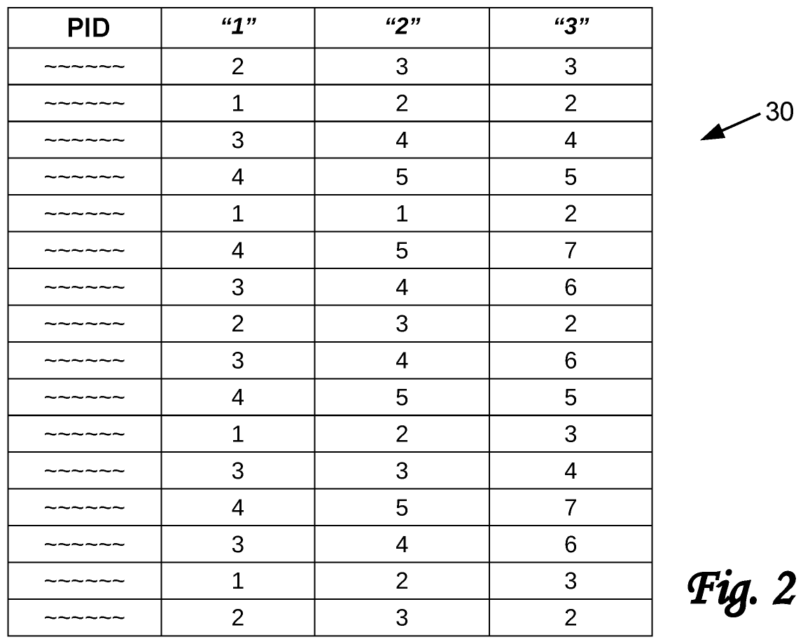

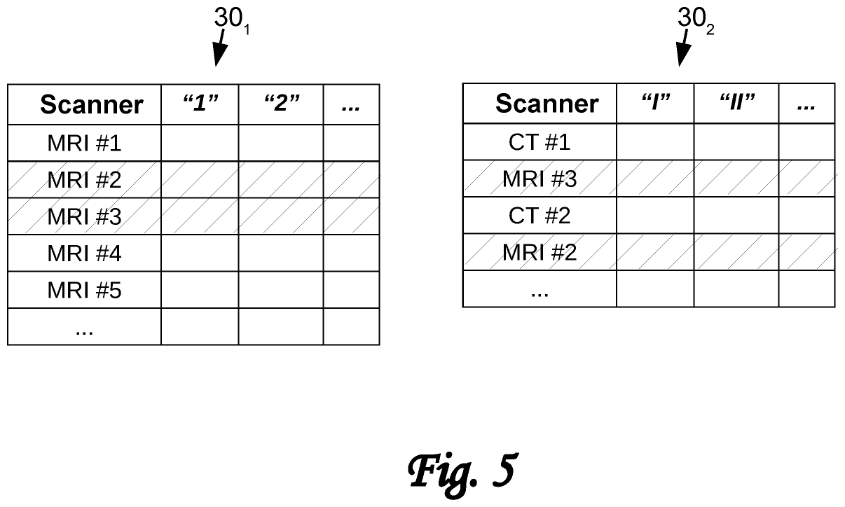

The received data are received by a data aggregator 28 which formats the received data as at least one table 30. In the following, a single table is assumed, for example having the format of the illustrative table 30 of FIG. 2. (FIG. 5, discussed later herein, describes extension of the approach to performing data checking on an illustrative two tables). The data aggregator 28 may perform various operations on the received data, such as checking data type (based on a priori knowledge of the expected data type, or based on a difference in data type of one datum compared with other similarly situated data), checking whether data falls within an a priori defined range, and so forth. The data aggregator 28 may also perform various processing such as data type conversion, organizing the data into the table 30, or so forth.

With continuing reference to FIG. 1, a pairwise columns comparator 32 is implemented by the computer 12 running the instructions read from the storage medium 14 in order to perform a (further) data quality check. The pairwise columns comparator 32 performs pairwise comparisons of columns of the table 30 to associate a first column and a second column with a one-to-N relationship, such as a one-to-one relationship or a one-to-many relationship. This is done by “assuming” the one-to-N relationship holds and detecting violations of the one-to-N relationship for data of the first and second columns. For example, if at decision block 38 it is determined that a count of the violations of the one-to-one relationship for the first and second columns is less than a threshold, then it is concluded that the one-to-one relationship at least mostly holds (that is, holds to the extent that the count of violations is less than the threshold), and the violations 40 of the now-concluded one-to-one relationship are taken as possible errors. Similarly, if at decision block 48 it is determined that a count of the violations of the one-to-many relationship for the first and second columns is less than a threshold, then it is concluded that the one-to-many relationship at least mostly holds (that is, holds to the extent that the count of violations is less than the threshold), and the violations 50 of the now-concluded one-to-many relationship are taken as possible errors. (Note that in the foregoing, “first” column and “second” column do not in general denote the ordinal first and second columns in the table, but rather denote two selected columns for which a one-to-one and/or one-to-many relationship is to be tested).

The resulting violations 40, 50 (if any) are taken to be possible errors. In one approach, these possible errors are presented to a user via a user interface (UI) 52 presented on an illustrative desktop or notebook computer 54 having a display 56 and one or more user input devices 58 (such as an illustrative keyboard and trackpad, and/or a mouse, trackball, touch-sensitive overlay of the display 56, various combinations thereof, and/or so forth). For example, possible data errors corresponding to the violations 40, 50 of the one-to-N relationship for data of the first and second columns may be indicated, e.g. displayed, on the display 56. In one approach, indication is displayed on the display 56 that the violation is not consistent with the one-to-N relationship. Instead of the illustrative desktop or notebook computer 54, the user interface 52 may be presented on a cellular telephone (i.e. cellphone), tablet computer, or any other user interfacing device having a display or other output component for presenting the possible errors (these are the violations 40, 50) and one or more user input devices for receiving input from the user. Via the UI 52, the user may correct the error by replacing the current value with a new value, or may delete the value entirely (or replace it with some “null” value if deletion is impermissible); or, the user may confirm that the present value is in fact correct.

Data 60 which are not violations, together with data 62 that are violations and have been reviewed and confirmed or corrected or nulled or otherwise remediated by the user via the US 52, are collected and stored in a database on a non-transitory storage medium 64 (for example a hard disk or other magnetic storage medium, a solid state drive or other electronic storage medium, an optical disk or other optical storage medium, a RAID, and/or so forth). The format in which the data are stored is application dependent. As an illustrative example, in a machine logging application (e.g. for the medical imaging device 24), the table 30 is suitably stored in a machine or service log of a medical imaging device, and the possible data errors are indicated as possible machine or service log errors. As another illustrative example, in a patient data recordation application, the table 30 is suitably stored in an electronic medical record (EMR), and the possible data errors are indicated as possible patient data errors. These are merely illustrative examples, and the disclosed pairwise columns-based data consistency checking may be employing in numerous other medical and non-medical electronic data processing tasks.

It should be noted that the non-transitory storage medium 64 may be the same as, different from, or partially overlapping with, the non-transitory storage medium 14 storing the instructions which are readable and executable by the electronic processor 12 to implement the electronic data processing system 10. Similarly, in some embodiments the computer 54 may be also used as a data entry computer 20.

In the following, some additional aspects of the electronic data processing system 10, and of the data quality checking performed by the pairwise columns comparator 32 in particular, are described.

The basic idea of the pairwise columns comparator 32 is to infer directly from the data stored in each pair of columns in a table whether or not there is a one-to-one or a one-to-many relation between them, approximately. A test is used to determine to what extent the relation of the data stored in the two columns (designated without loss of generality here as a “first” column and a “second column”) is one-to-one or one-to-many. And if it is likely that the relation is one-to-one or one-to-many (determined at respective blocks 38, 48), then the violations of this relation (e.g., violations 40, 50) are flagged as a potential data quality issue. These potential data quality issues 40, 50 can be indicated as alerts in a dashboard UI 52, so that subject matter experts can examine whether indeed these are data quality issues or whether the suspected relation is not valid. If the latter is the case, then this will be remembered by the data quality checker 32, so that in a next evaluation these alerts are not raised again.

To more precisely describe some preferred embodiments, the following notation is employed herein. Let T be an n×m database table of entries (table 30), with n rows and m columns. Entry t(i,j) denotes the entry in row i and column j. Typically, a table stores information on entities of the same type (e.g. the “type” may be a patient, or a medical imaging device, or so forth). Each row corresponds to information on a single entity (a single medical imaging device, or a single patient, . . . ) and each column corresponds to a specific property (or data field or so forth) of such entities (type, age, . . . ). The pairwise columns comparator 32 is comparing a “first” column j and a second column j′ (where again “first” and “second” are arbitrary labels for the chosen two columns to be compared, and do not denote ordinal position of the columns in the table). Using this notation, two columns j and j′ have a one-to-one relationship if and only if:

for any pair i and i′: t(i,j)=t(i′,j) if and only if t(i,j′)=t(i′,j′) (1)

Said another way, the one-to-one relationship is defined for the first column j and the second column j′ as:

t(i,j)=t(i′,j) if and only if t(i,j′)=t(i′,j′) (2)

where i and i′ are different rows of the table. For example, in a table with information on countries, the columns with country name and capital name will have a one-to-one relation.

Two columns j and j′ have a one-to-many relationship if and only if:

for any pair i and i′:t(i,j′)=t(i′,j′) implies that t(i,j)=t(i′,j) (3)

but not necessarily the other way around. Said another way, the one-to-many relationship is defined for the first column j and the second column j′ as:

if t(i,j′)=t(i′,j′) then t(i,j)=t(i′,j) (4)

where i and i′ are different rows of the table. For example, in a table with information on persons, there is a one-to-many relation from the column with country name to the column with city name. If two persons live in the same city, then their country will also be the same, assuming for the sake of argument that city names are unique in the world.

To infer a possible one-to-one or one-to-many relation (identified generically herein as one-to-N relation, where N can be “one” or “many”), an undirected, weighted bipartite graph G=(Vⱼ,V_{j′},E) is used, where Vⱼ denotes a node set on the left side of the graph defining the values that occur in column j, and V_{j′} denotes a node set on the right side of the graph defining the values that occur in column j′. The set E of edges only connect a node from Vⱼ with a node from V_{j′}, i.e., there are no edges between two nodes from Vⱼ and no edges between two nodes from V_{j′}. There is an undirected edge e=(u, v)∈E between u∈Vⱼ and v∈V_{j′} if and only if the corresponding values occur together in one or more rows of table T. Both nodes and edges have weights whose value is an integer. More particularly, each node u∈Vⱼ has a weight w(u) denoting the number of times that the corresponding value occurs in column j. Each node v∈V_{j′}, has a weight w(v) denoting the number of times that the corresponding value occurs in column j′. Each edge e=(u, v) has a weight w(e), also represented here as weight w(u, v), denoting the number of rows in which the values corresponding to u and v co-occur, where by definition w(u, v)=0 if (u, v)∉E.

Put another way, the weighted bipartite graph has: a first (i.e. left) part comprising nodes (Vⱼ) representing values occurring in the first column (j) weighted by counts (i.e. weights w(u)) of the occurrences of the respective values in the first column; a second (i.e. right) part comprising nodes (V_{j′}) representing values occurring in the second column (j′) weighted by counts (i.e. weights w(v)) of the occurrences of the respective values in the second column, and edges (E) connecting nodes of the first part and nodes of the second part having weights (w(u, v)) corresponding to counts of co-occurrences of the respective values represented by the connected nodes in the respective first and second columns. In one approach for the pairwise columns comparison, such a weighted bipartite graph representing the first and second columns is generated; and violations of the one to N relationship for the data of the first and second columns are detected using the bipartite graph.

It is not difficult to see that for each node u∈Vⱼ the following holds:

$$w ( u ) = {\sum }_{v ∈ {V}_{j ′}} w ( u , v ) \qquad ( 5 )$$

And that for each node v∈V_{j′} the following holds:

$$w ( v ) = {\sum }_{u ∈ {V}_{j}} w ( u , v ) \qquad ( 6 )$$

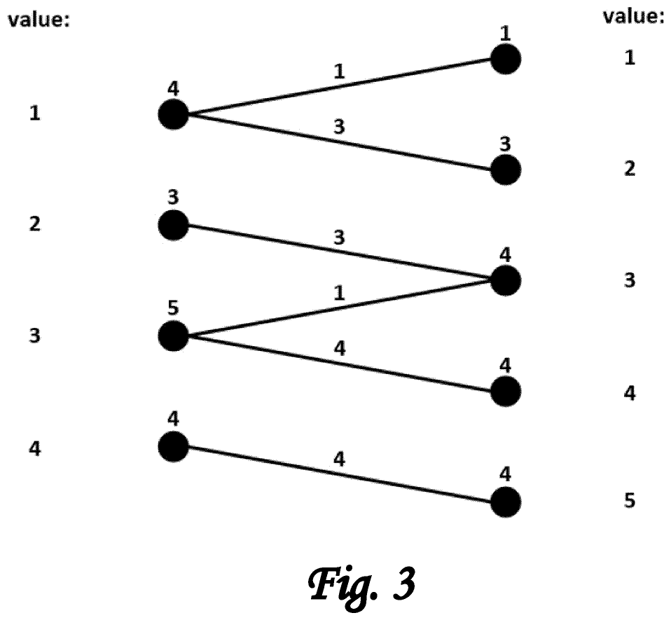

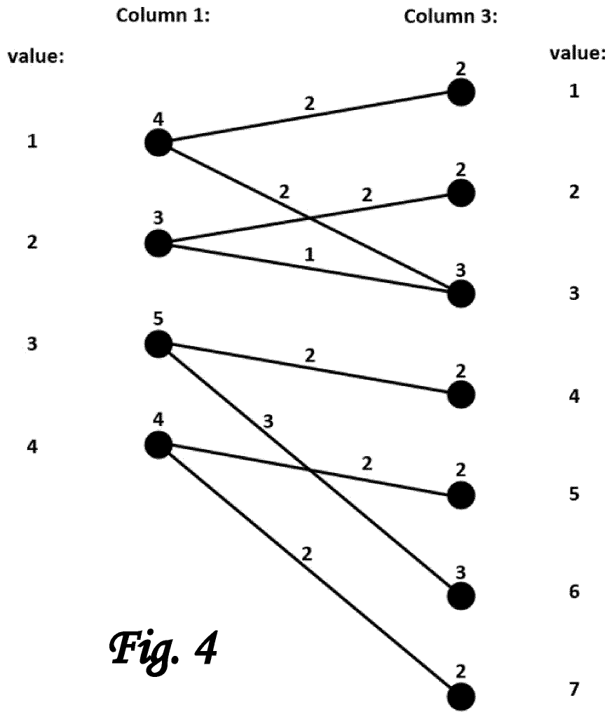

With reference now to FIGS. 2-4, consider as an example the table 30 shown in FIG. 2, with 16 rows and 3 columns. FIG. 2 also shows column headers (i.e. column labels): “PID” (representing “Patient Identification”); “1”, “2”, and “3”. These should be taken as merely illustrative labels without informational significance. The weighted bipartite graph representing the relation between the values in column “1” and column “2” is shown in FIG. 3; while, the weighted bipartite graph representing the relation between the values in column “1” and column “3” is shown in FIG. 4. In other words, FIG. 3 shows the weighted bipartite graph for the first column being column “1” and the second column being column “2”; and FIG. 4 shows the weighted bipartite graph for the first column being column “1” and the second column being column “3”.

Furthermore, let the degree of a node of a weighted bipartite graph be defined as the number of edges that are connected to the node. If a null value (usually interpreted as a missing value) is interpreted as just one of the possible entry values, then the degree of each node is at least one, since each value occurring in one of the columns must have at least one matching value in the other column.

The following definitions are employed: a fully one-to-one relation between columns j and j′ is present if all nodes in graph G have a degree one. A fully one-to-many relation from column j to column j′ is present if all nodes in V_{j′} (i.e., the node set corresponding to values in column j′) have a degree one.

To infer approximate (i.e. mostly) one-to-one or one-to-many relations, let m(u) denote the maximum weight of an edge connected to node u∈V. For node v∈V_{j′}, let m(v) denote the maximum weight of an edge connected to node v∈V_{j′}. By definition, m(u)≤w(u) and m(v)≤w(v) and for a fully one-to-one relation it follows that m(u)=w(u) and m(v)=w(v) for all u∈Vⱼ and v∈V_{j′}. Furthermore, for fully one-to-many relations from column j to column j′ it follows that m(v)=w(v) for all v∈V_{j′}.

For approximately one-to-one or one-to-many relations, the number of values of the total n values that occur in a single column, say column j, that violate the relation can be counted by the following expression, called the number of violations nov(j) of column j:

nov(j)=Σ_{u∈V}_{ⱼ}(w(u)−m(u)) (7)

Likewise, for column j′, the count of violations is given by:

nov(j′)=Σ_{v∈V}_{_{j′}}(w(v)−m(v)) (8)

For a table with n rows, for both columns j, j′ the following respectively hold: nov(j)<n and nov(j′)<n. For an approximate (i.e. mostly) one-to-one relation, the number of violations for both columns should be counted, and the relative (i.e. normalized) number of violations can be defined by:

$$\frac{nov ( j ) + nov ( {j}^{′} )}{2 n} \qquad ( 9 )$$

where the value ½(nov(j)+nov(j′)) is normalized by the total number of rows (n) in the table 30. For an approximate (i.e. mostly) one-to-many relation from column j to column j′, it is not required that the nodes in U all have a degree one, so only the violations for column j′ are relevant. Hence for a one-to-many relation the relative number of violations is given by:

$$\frac{nov ( {j}^{′} )}{n} \qquad ( 10 )$$

where the value nov(j′) is normalized by the total number of rows (n) in the table 30.

To quantitatively define a “mostly” one-to-one relation, a threshold T₁₋₁ is specified, and there is a (mostly) one-to-one relation between columns j and j′ if and only if:

$$\frac{nov ( j ) + nov ( {j}^{′} )}{2 n} \leq {T}_{1 - 1} \qquad ( 11 )$$

In a similar fashion, to quantitatively define a “mostly” one-to-many relation, a threshold T_{1-many} is specified, and there is a (mostly) one-to-many relation between columns j and j′ if and only if:

$$\frac{nov ( {j}^{′} )}{n} \leq {T}_{1 - many} \qquad ( 12 )$$

In Equations (11) and (12) different thresholds T₁₋₁ and T_{1-many} are employed. However, these may optionally be the same value, i.e. T₁₋₁=T_{1-many} is a possibility. The above approach to identify approximate one-to-one and one-to-many relations is an illustrative embodiment. Alternative embodiments can be employed for quantitatively defining (mostly) one-to-one or (mostly) one-to-many relations, such as, for example, counting the average degree (minus one) of nodes to establish a relative number of violations.

By way of illustration, for the weighted bipartite graph for columns 1 and 2, shown in FIG. 3, it is determined that for column “1”, the number of violations of the one-to-one relation is nov(1)=2, as two nodes have a contribution of one, for w(uₖ)−m(uₖ). Additionally, for column “2”, the number of violations of the one-to-one relation is nov(2)=1. Since the number of rows n is equal to 16, the relative number of violations for a one-to-one relation would be 3/32=0.09375. If the threshold T₁₋₁ is chosen equal to 0.1, then the relation between columns “1” and “2” is assumed to be one-to-one, and the two rows with value combinations (1,1) and (3,3), respectively, are flagged as potential data quality issues (i.e., are flagged as one-to-one relationship violations 40, using the framework of FIG. 1).

Similarly, for the weighted bipartite graph for columns 1 and 3, shown in FIG. 4, for column “1” the number of violations of the one-to-one relation is nov(1)=7 and for column “3” the number of violations of the one-to-one relation is nov(3)=1. Hence, the relative number of violations for a one-to-one relation would be 8/32=0.25. If the threshold T₁₋₁ is chosen equal to 0.1, then the relation between columns “1” and “3” is discarded as being a one-to-one relationship. However, for a one-to-many relationship, only the number of violations for column “3” are counted. Hence, for a one-to-many relationship the result is a relative number of violations of 1/16=0.0625. For T_{1-many}=0.1, it is then concluded that the relation between columns “1” and “3” is mostly one-to-many, and the single row with value combination (2,3) is then flagged as potential data quality issue (i.e., is flagged as a one-to-many relationship violation 50, using the framework of FIG. 1).

In this way, given a number of pairs of columns for which a one-to-one or a one-to-many relation holds approximately, the violations 40, 50 can be identified and raised as possible data quality issues. In a dashboard (or other UI 52), the suspected type of relation that holds approximately can be marked, so that a subject matter expert can indicate whether or not this relation is true. And if so, the violations can be inspected by the subject matter expert and explicitly marked as data quality issues. Note that in this way, a subject matter expert is only bothered with questions about potential one-to-one or one-to-many relations, whenever there are potential exceptions for these relations. In this way, data quality issues can be identified in an early stage so that actions can be taken to further pollute the contents of the database.

In some embodiments, the UI 52 optionally presents one or more solutions to identified data quality issues (i.e., solutions for the detected violations 40, 50). For example, if it has been established that there is (likely) to be a one-to-one relation between columns j and j′, then if value uₖ in column j almost always co-occurs with value vₗ in column j′, then for the few exceptions where it co-occurs with value vₘ in column j′ the UI 52 suitably suggests to change values vₘ into vₗ for the cases where they co-occur with uₖ in column j. Similar suggestions can be made for one-to-many relations.

Furthermore, once a relation has been confirmed by a subject matter expert, it can be treated with high priority in subsequent data quality checks.

With reference now to FIG. 5, it is further noted that this analysis can also be carried out for two columns from different tables. In that case, the number of rows for both tables may be different and the definitions of the relative number of violations has to be adapted correspondingly. Illustrative FIG. 5 shows an example in which the at least one table 30 includes a first table 30₁ and a second table 30₂. In this example, the tables store data relating to medical imaging devices. First table 30₁ stores information relating to magnetic resonance imaging (MRI) devices numbered #1-#5. Second table 30₂ stores information relating to various medical imaging devices including both MRI devices and computed tomography (CT) devices. Consider a pairwise column comparison across the tables 30₁, 30₂ in which column “1” of first table 30₁ and column “II” of second table 30₂ are to be compared. The fact that these columns “1” and “II” are in different tables is not a difficulty so long as corresponding rows of the two tables can be identified, for example based on an index value identifying the entity. For example, in tables 30₁, 30₂ the corresponding rows are the two rows indexed by “MRI #2” and the two rows indexed by “MRI #3”, as these rows can be identified as having correspondence in the two table due to the common index “MRI #2” or “MRI #3”. The corresponding rows are shaded in FIG. 5. It will be appreciated that, for example, when applying the one-to-one and one-to-many relationships of Equations (1)-(4) for pairwise comparison of columns “1” and “II”, this can be done so long as only those corresponding rows (shaded rows in FIG. 5) are considered. This is because only for corresponding rows are both values t(i, “1”) and t(i, “II”) defined. Hence, the disclosed approach can be employed if there are enough corresponding rows in the two tables for the one-to-one or one-to-many relationships to be defined with sufficient statistics. If this is the case, then the pairwise comparison can be employed to determine if (for the illustrative example) the values of columns “1” and “II” exhibit, e.g., a one-to-many relationship. If so, then any corresponding rows of the two tables for which t(i, “1”) and t(i, “II”) violate Equation (3) (or equivalently, Equation (4)) is flagged as a violation. For those rows of the first table 30₁ that do not have corresponding rows in the second table 30₂, the one-to-many relationship check is simply not performed, and vice versa for rows of second table 30₂ with no correspondence in first table 30₁.

Additionally, the approach is modified when comparing columns across tables by the normalization factor. As noted with reference to Equations (9) and (10), the count of the violations is normalized by the total number of rows (n) in the table 30. When comparing columns across two tables 30₁, 30₂, on the other hand, the count of the violations is normalized by the total number of corresponding rows of the first and second tables 30₁, 30₂. For example, the normalization factor is 2 in the example of FIG. 5 since there are two corresponding rows (namely the corresponding rows with index “MRI #2” and the corresponding rows with index “MRI #3”).

As a further variant, the disclosed approach can be extended to more than two columns. For example, column pairs can be compared to a third column to determine if there is a 1-1 or 1-N correlation. Such second order analysis might help further narrow the exceptions, find new exceptions, or help in root cause analysis methodologies. Hence, this second order analysis entails performing further comparisons of columns of at least one table 30, 30₁, 30₂ to associate a first pair of columns and a third column with a one-to-N relationship (e.g., a 1-1 relationship or a 1-many relationship) by detecting violations of the one-to-N relationship for data of the first pair of columns and the third column and determining that a count of the violations of the one-to-N relationship for the first pair of columns and the third column is less than a threshold. Various approaches could be used to define a one-to-N relationship in this context. In one approach, a match can be found for a row if a 1-1 (or 1-N) relation holds between either column of the pair and the third column. In another approach, a match can be found for a row if a 1-1 (or 1-N) relation holds between each column of the pair and the third column.

The invention has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. An electronic data processing system comprising:
an electronic processor; and
a non-transitory storage medium storing instructions readable and executable by the electronic processor to perform an electronic data processing method including:
performing pairwise comparisons of columns of at least one table to associate a first column and a second column with a one-to-N relationship by detecting violations of the one-to-N relationship for data of the first and second columns and determining that a count of the violations of the one-to-N relationship for the first and second columns is less than a threshold; and
indicating, on a display, possible data errors corresponding to the violations of the one-to-N relationship for data of the first and second columns;
wherein the performing and the indicating are performed for the one-to-N relationship being a one-to-one relationship and/or for the one-to-N relationship being a one-to-many relationship.

2. The electronic data processing system of claim 1 wherein the comparison of the first and second columns is performed by operations including:
generating a weighted bipartite graph representing the first and second columns; and
detecting the violations of the one-to-N relationship for the data of the first and second columns using the bipartite graph.

3. The electronic data processing system of claim 2 wherein the weighted bipartite graph has:
a first part comprising nodes representing values occurring in the first column weighted by counts of the occurrences of the respective values in the first column,
a second part comprising nodes representing values occurring in the second column weighted by counts of the occurrences of the respective values in the second column, and
edges connecting nodes of the first part and nodes of the second part having weights corresponding to counts of co-occurrences of the respective values represented by the connected nodes in the respective first and second columns.

4. The electronic data processing system of claim 1 wherein the performing and the indicating are performed at least for the one-to-N relationship being a one-to-one relationship defined for the first column j and the second column j′ as:
t(i,j)=t(i′,j) if and only if t(i,j′)=t(i′,j′)
where i and i′ are different rows of the at least one table.

5. The electronic data processing system of claim 1 wherein the performing and the indicating are performed at least for the one-to-N relationship being a one-to-many relationship defined for the first column j and the second column j′ as:
if t(i,j′)=t(i′,j′) then t(i,j)=t(i′,j)
where i and i′ are different rows of the at least one table.

6. The electronic data processing system of claim 1 wherein the indicating includes, for each violation of the one-to-N relationship, displaying on the display an indication that the violation is not consistent with the one-to-N relationship.

7. The electronic data processing system of claim 1 wherein the at least one table consists of a single table and the count of the violations of the one-to-N relationship in the first and second columns is normalized by the total number of rows of the single table.

8. The electronic data processing system of claim 1 wherein the at least one table consists of first and second tables, the first column is in the first table, the second column is in the second table, and the count of the violations of the one-to-N relationship in the first and second columns is normalized by the total number of corresponding rows of the first and second tables.

9. The electronic data processing system of claim 1 wherein the electronic data processing method further includes:
storing the at least one table in a machine or service log of a medical imaging device;
wherein the indicating includes indicating the possible data errors as possible machine or service log errors.

10. The electronic data processing system of claim 1 wherein the electronic data processing method further includes:
storing the at least one table in an electronic medical record (EMR);
wherein the indicating includes indicating the possible data errors as possible patient data errors.

11. The electronic data processing system of claim 1 wherein the electronic data processing method further includes:
performing further comparisons of columns of at least one table to associate a first pair of columns and a third column with a one-to-N relationship by detecting violations of the one-to-N relationship for data of the first pair of columns and the third column and determining that a count of the violations of the one-to-N relationship for the first pair of columns and the third column is less than a threshold.

12. A non-transitory storage medium storing instructions that are readable and executable by an electronic processor to perform an electronic data processing method comprising:
identifying a first column and a second column of at least one table which have a one-to-one or one-to-many relationship with fewer than a threshold number of violations of the one-to-one or one-to-many relationship; and
indicating, on a display, possible data errors in the at least one table corresponding to the violations of the one-to-one or one-to-many relationship.

13. The non-transitory storage medium of claim 12 wherein the identifying includes:
identifying the first column and the second column of the at least one table which have a one-to-one relationship with fewer than a threshold number of violations of the one-to-one relationship.

14. The non-transitory storage medium of claim 13 wherein the one-to-one relationship is defined for the first column j and the second column j′ as:
t(i,j)=t(i′,j) if and only if t(i,j′)=t(i′,j′)
where i and i′ are different rows of the at least one table.

15. The non-transitory storage medium of claim 12 wherein the identifying includes:
identifying the first column and the second column of the at least one table which have a one-to-many relationship with fewer than a threshold number of violations of the one-to-many relationship.

16. The non-transitory storage medium of claim 15 wherein the one-to-many relationship is defined for the first column j and the second column j′ as:
if t(i,j′)=t(i′,j′) then t(i,j)=t(i′,j)
where i and i′ are different rows of the at least one table.

17. The non-transitory storage medium of claim 12 wherein the identifying includes:
generating a weighted bipartite graph representing the first and second columns; and
detecting the violations of the one-to-one or one-to-many relationship for the data of the first and second columns using the bipartite graph.

18. The non-transitory storage medium of claim 17 wherein the weighted bipartite graph has:
a first part comprising nodes representing values occurring in the first column weighted by counts of the occurrences of the respective values in the first column,
a second part comprising nodes representing values occurring in the second column weighted by counts of the occurrences of the respective values in the second column, and
edges connecting nodes of the first part and nodes of the second part having weights corresponding to counts of co-occurrences of the respective values represented by the connected nodes in the respective first and second columns.

19. The non-transitory storage medium of claim 12 wherein the electronic data processing method further comprises:
displaying, on the display, a user interface whereby a user accepts or rejects each indicated violation of the one-to-one or one-to-many relationship.

20. An electronic data processing method comprising:
identifying, using an electronic processor, a first column j and a second column j′ of at least one table which have a mostly one-to-N relationship; and
indicating, on a display, possible data errors in the at least one table corresponding to violations of the identified one-to-N relationship;
wherein the one-to-N relationship is a one-to-one relationship defined as:
t(i,j)=t(i′,j) if and only if t(i,j′)=t(i′,j′)
or a one-to-many relationship defined as:
t(i,j)=t(i′,j) if and only if t(i,j′)=t(i′,j′)
where i and i′ are different rows of the at least one table.

21. The electronic data processing method of claim 20 wherein the identifying includes generating a weighted bipartite graph representing the first and second columns, wherein the weighted bipartite graph has:
a first part comprising nodes representing values occurring in the first column weighted by counts of the occurrences of the respective values in the first column,
a second part comprising nodes representing values occurring in the second column weighted by counts of the occurrences of the respective values in the second column, and
edges connecting nodes of the first part and nodes of the second part having weights corresponding to counts of co-occurrences of the respective values represented by the connected nodes in the respective first and second columns.
